(19) United States
(12) Patent Application Publication
(10) Pub. No.: US 2024/0190924 A1
(43) Pub. Date: Jun. 13, 2024

(54) Synthesis of Gamma Peptide Nucleic Acid Monomers and Oligomers, and Applications Therefor

(71) Applicant: Carnegie Mellon University, Pittsburgh, PA (US)

(72) Inventors: Danith H. Ly, Pittsburgh, PA (US); Shivaji A. Thadke, Pittsburgh, PA (US); Isha Dhami, Pittsburgh, PA (US)

(21) Appl. No.: 18/279,687

(22) PCT Filed: Mar. 1, 2022
(86) PCT No.: PCT/US22/18300

§ 371 Date: Aug. 31, 2023

Related U.S. Application Data
(60) Provisional application No. 63/154,840, filed on Mar. 1, 2021.

(51) Int. Cl. C07K 14/00

(52) U.S. Cl. CPC C07K 14/003

(57) ABSTRACT

Provided herein are methods of preparing gamma-peptide nucleic acid monomers, and methods of synthesizing gamma-peptide nucleic acid oligomers.

### CROSS REFERENCE TO RELATED APPLICATIONS

This application is the United States national phase of International Application No. PCT/US22/18300, filed Mar. 1, 2022, and claims priority to U.S. Provisional Application, No. 63/154,840, filed Mar. 1, 2021, the disclosures of which are hereby incorporated by reference in their entireties.

Originally developed by Peter Nielsen and coworkers in the early 1990's, peptide nucleic acids (PNAs) have emerged as a promising class of nucleic acid mimics for biological and biomedical applications because of their tight and specific binding, and resistance to enzymatic degradation by proteases and nucleases. Building on this initial molecular scaffold, it has been shown that PNAs, which do not adopt a well-defined conformation, can be preorganized into either a left-handed (LH) or right-handed (RH) helical motif by installing an appropriate stereogenic center at the gamma backbone (FIGS. 1A and 1B). The former is orthogonal to RHγPNA, DNA, and RNA in recognition, while the latter exhibits unusually strong binding affinity for DNA and RNA. RHγPNA is the only class of oligonucleotide molecules developed to date that can invade double helical DNA under physiological temperature without sequence restriction, a feature that has been successfully exploited in gene editing.

The recognition orthogonality and the translational capability of the (LHγPNA)/(PNA)/(RHγPNA) system has generated considerable interest in its applications in molecular computing and cell therapy. With respect to the latter, the utilization of relatively short, orthogonal LHγPNAs for programming cell-cell interaction is attractive, for instance, in directing chimeric antigen receptor T (CAR-T) cells to engage cancer cells, because of the relative ease of production, the close proximity of the two cell types required for effective cancer cell killing, and their inertness to hybridization with DNA or RNA upon internalization into the cell's nucleus and cytoplasm. However, with current design, the charge-neutral backbone imposes a major challenge for their handling and molecular manipulation. Due their poor water solubility, these molecules and the corresponding complexes are prone to aggregation and association with other macromolecules and surfaces in a nonspecific manner, making them inferior for molecular self-assembly. Likewise, when covalently attached to cells or other materials, they have a propensity to collapse onto the surface, and in the former case, are buried in the membrane, rendering them ineffective for hybridization with their complementary partners that are in solution or covalently attached to other extracellular components or material's surfaces.

There is a need for LHγPNA and RHγPNA monomers and oligomers containing strategically placed or fully-modified groups, such as phosphate, guanidine, and dihydroxypropyl gamma-side chain, as well as methods for easy modification of groups attached to the gamma side chain.

### SUMMARY

According to an aspect or embodiment, a method of making a peptide nucleic acid monomer is provided, comprising:

phosphorylating compound 1, where n is 1, 2, 3, or 4, R₁ is an amine-protecting group:

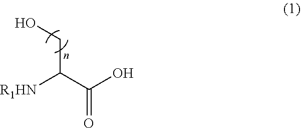

with trichlorophosphorus (PCl₃), phosphoramidous acid, N,N-bis(1-methylethyl)-, bis(phenylmethyl) ester, or 4,3-Benzodioxaphosphepin, 3-chloro-15-dihydro-, 3-oxide), R₂—OH, and R₃—OH, to produce compound 2, where R₂ and R₃ are, independently, H, benzyl

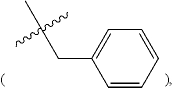 [SMILES: *Cc1ccccc1]

t-butyl, propionitrilyl

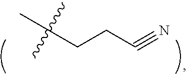

or 4-nitrophenylethylenyl

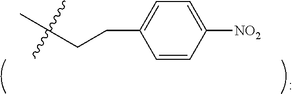

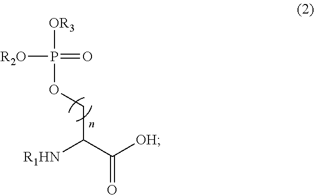

reducing compound 2 to produce compound 3:

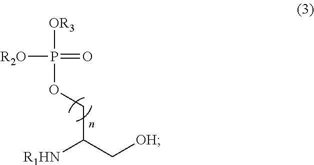

reacting compound 3 with Dess-Martin periodinane, followed by treating with NH₂CH₂C(O)OCH₃ and DCM, or reacting compound 3 with DMSO:TEA followed by treating with NH₂CH₂C(O)OCH₃, to produce compound 4:

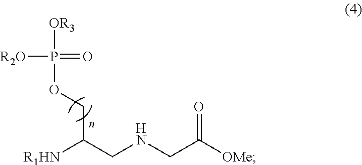

and
conjugating compound 4 with a nucleobase by reacting compound 4 with RCH₂C(O)OH, where R is a nucleobase in which α-amines of R are protected with an amine-protecting group to produce compound 5:

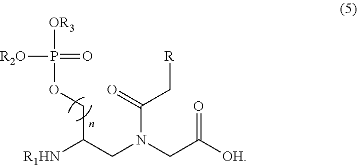

According to another aspect or embodiment, a method of making a peptide nucleic acid monomer is provided, comprising:

reducing compound 6:

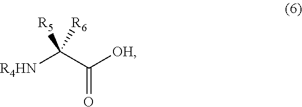

where R₄ is an amine-protecting group, and one of R₅ and R₆ is H, and the other of R₅ and R₆ is:

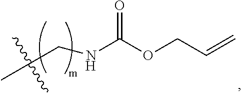

where m is 1, 2, 3, or 4, to produce compound 7:

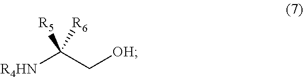

reacting compound 7 with Dess-Martin periodinane, followed by treating with NH₂CH₂C(O)OCH₃ (methyl glycinate), or reacting compound 7 with DMSO:TEA followed by treating with NH₂CH₂C(O)OCH₃, to produce compound 8:

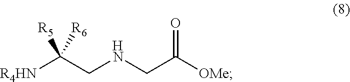

conjugating compound 8 with a nucleobase by reacting compound 8 with RCH₂C(O)OH, where R is a nucleobase in which α-amines of R are protected with an amine-protecting group to produce compound 9:

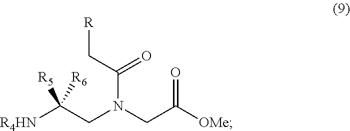

and
removing the terminal methyl group of 9 to produce compound 10:

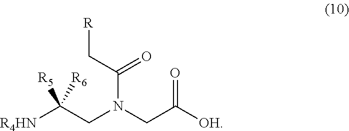

According to another aspect or embodiment, a compound having the structure:

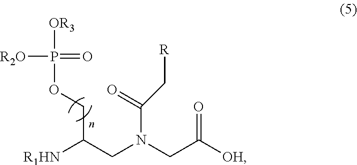

or a salt thereof is provided, along with enantiopure compositions comprising a stereoisomer of that compound, as well as a racemic mixture of stereoisomers of that compound in any relative proportion. A peptide nucleic acid comprising a residue (one or more residues) of the monomer also is provided in aspects or embodiments.

According to another aspect or embodiment, a solid-phase peptide nucleic acid synthesis method is provided comprising sequentially extending a sequence of peptide nucleic acid monomers from a substrate to produce a peptide nucleic acid comprising the sequence of peptide nucleic acid monomers and wherein the sequence of peptide nucleic acid monomers comprising at least one residue of a γPNA monomer having the structure:

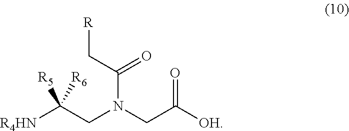

where R₄ is an amine-protecting group, and one of R₅ and R₆ is H, and the other of R₅ and R₆ is

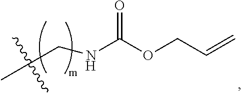

where m is 1, 2, 3, or 4,
deprotecting the peptide nucleic acid to remove the allyloxycarbonyl group from R₅ and R₆, e.g., using Pd(PPh₃)₄ (palladium-tetrakis(triphenylphosphine)) or PhSiH₃ (phenylsilane), and linking the unprotected amine of R₅ or R₆ with an amine-reactive compound to modify the peptide nucleic acid; and
cleaving the peptide nucleic acid from the substrate

According to another aspect or embodiment, a peptide nucleic acid monomer, having the structure:

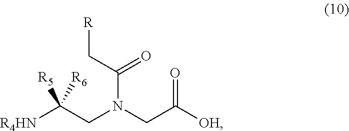

where one of R₅ and R₆ is H, and the other of R₅ and R₆ is:

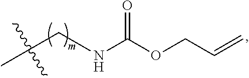

where m is 1, 2, 3, or 4. IN another aspect or embodiment, a γPNA is provided, comprising a monomer residue of that peptide nucleic acid monomer.

According to another aspect or embodiment, a method of making a peptide nucleic acid monomer is provided, comprising:

adding an amine protecting group to the terminal amine and adding a 4,4′-dimethoxytrityl (DMT) group to the primary hydroxyl group of compound 11:

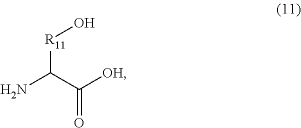

where R₁₁ is —(CH₂)ₙ—, and n is 1-4,
to produce compound 12, where R₁₂ is an amine-protecting group;

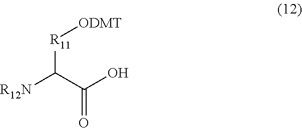

reducing compound 12 and reacting compound 12 with Dess-Martin periodinane followed by methyl glycinate to produce compound 13:

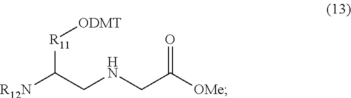

conjugating compound 13 with a nucleobase by reacting compound 13 with RCH₂C(O)OH, where R is a nucleobase in which α-amines of R are protected with an amine-protecting group and removing the methyl group from the terminal carboxymethyl group to produce compound 14:

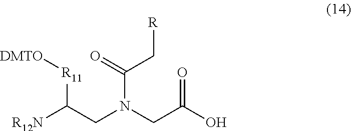

phosphorylating the DMT-protected oxygen with:
dichloroacetic acid,

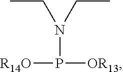

tetrazole, and I₂, to produce compound 15:

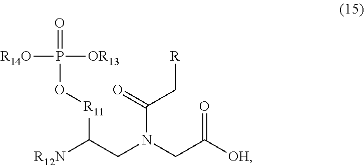

where R₁₃ and R₁₄ are, independently, H, benzyl, t-butyl, propionitrilyl, or 4-nitrophenylethylenyl.

According to another aspect or embodiment, a solid-phase peptide nucleic acid synthesis method is provided. comprising sequentially extending a sequence of peptide nucleic acid monomers from a substrate to produce a peptide nucleic acid comprising the sequence of peptide nucleic acid monomers and wherein the sequence of peptide nucleic acid monomers comprising at least one residue of a γPNA monomer having the structure:

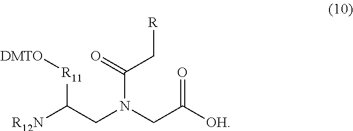

where R₁₁ is —(CH₂)ₙ—, and n is 1-4 and R₁₂ is an amine-protecting group; and
phosphorylating the DMT-protected oxygen with:
dichloroacetic acid,

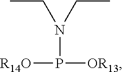

tetrazole, and I₂; and
cleaving the peptide nucleic acid from the substrate.

### BRIEF DESCRIPTION OF THE DRAWINGS

The terms Fig., Figs., Figure, and Figures are used interchangeably in the specification to refer to the corresponding figures in the drawings.

FIGS. 1A and 1B: FIG. 1A shows a generic structure of PNA, with a being (achiral) PNA, b being right-handed gamma PNA (RH-γPNA), and c being left-handed gamma PNA (LH-γPNA), where X is not H. γ identifies the γPNA gamma carbon. FIG. 1B shows Chemical structures of exemplary left- and right-handed phosphate-containing gamma peptide nucleic acid (PγPNA) monomers. R₁: amino protecting groups; R₂ and R₃: H or alcohol protecting groups; n: 1-4 (sidechain linker), B: nucleobases (natural or synthetic). Non-limiting examples of R₁: Boc, Fmoc; of R₂ and R₃: H, Bn (benzenyl), tBu (tert-butyl), CH₂CH₂CN, 4-nitrophenylethyl; of n: 1 (serine sidechain) and 2 (homoserine sidechain); and of B: adenine (A), cytosine (C), guanine (G), thymine (T), uridine (U), and unnatural nucleobases (see, e.g., FIGS. 2A and 2B).

FIGS. 2A and 2B: Selected examples of nucleobases. (A) Natural nucleobases, and unnatural nucleobases containing (B) monofacial and (C) bifacial recognition elements. R₄ is an amine-protecting group, such as Boc, which may be removed (deprotected) after PNA oligomer synthesis.

FIG. 3: Exemplary synthesis scheme for left-handed, serine-derived, phosphate-containing gamma peptide nucleic acid (LHPγPNA) monomers containing natural nucleobases (A, C, G, T).

FIG. 4: Exemplary scheme for solid-phase synthesis of phosphate-containing γPNA oligomers.

FIG. 5: Exemplary scheme for synthesis of right-handed (RH, 10a-d) and left-handed (LH, 10′a-d), lysine-derived, gamma peptide nucleic acid monomers containing natural nucleobases (A, C, G, T).

FIG. 6: Exemplary scheme for synthesis of the right-handed (RH, 15a-d) and left-handed (LH, 15′a-d), phosphorylated, gamma peptide nucleic acid monomers containing natural nucleobases (A, C, G, T). B is a nucleobase, except in NaBH₄ and NaB(OAc)₃H, where it refers to boron.

FIG. 7: Exemplary on-resin (solid-phase) synthesis of gamma peptide nucleic acid oligomers containing phosphate (P4a), guanidine (P4b), and dihydroxypropyl (P4c) chemical functionalities at the gamma backbone position.

FIG. 8 provides sequences of synthesized PNA's and control RNAs (SEQ ID NOS: 1 and 2) evaluated in Example 5.

FIG. 9: Chiral HPLC profiles of the right-handed (9c) and left-handed (9′c) methyl ester cytosine-monomers.

FIGS. 10A and 10B: (FIG. 10A) HPLC profiles of the P4 oligomer series containing Alloc-protecting group [P4 (Alloc)] and after its removal [P4 (NH₂)]. (FIG. 10B) HPLC profiles of P4a, P4b, and P4c.

FIG. 11: UV-melting profiles of PNA (P1) and gammaPNA (P2, P3a, P3b, P3c (upper), P4a, P4b, and P4c (lower)) oligomers with a complementary RNA strand (R1) in 1×PBS buffer (10 mM NaPi, pH 7.4). The concentration of each strand was 2.5 μM. Refer to FIG. 8 for the sequence compositions of the oligomers.

FIG. 12: The effects of sequence mismatches on the thermal stability of the gammaPNA-RNA duplexes. The samples were prepared in 1×PBS buffer, and the concentration of each strand was 2.5 μM. The nature of the mismatch for each group of PNA oligomers (P2, P4a, P4b, and P4c) is depicted in the R1b traces. Sequences are provided in FIG. 8.

FIG. 13: Circular dichroism (CD) profiles of the individual PNA (P1) and gammaPNA strands (P2, P4a, and P5a), along with the corresponding gammaPNA-gammaPNA duplexes (bottom). Buffer: 10 mM NaPi, 137 mM NaCl, 150 mM KCl, 2 mM MgCl₂, pH 7.4; strand concentration: 2.5 μM each.

FIG. 14: Photographs showing results of the gel-shift assays demonstrating recognition orthogonality. (a) Incorporation of the phosphate group at the gamma-backbone position enabled the characterization of their electrophoretic mobilities. (b-d) Demonstration of the recognition orthogonality of RNA (R1af), and the right-handed (P4af) and left-handed (P5aF) with the indicated complementary strands, respectively. In a, the concentration of each strand was 0.5 μM, and in b-d, equimolar concentrations (0.5 μM) of the indicated strands were prepared and incubated at 37° C. for 60 min, and separated on a 20% non-denaturing gel

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include, but are not limited to embodiments “consisting essentially of” and “consisting of” these stated elements or steps.

A polymer, such as a PNA, e.g., a γPNA, “comprises” or is “derived from” a stated monomer if that monomer is incorporated into the polymer. Thus, the incorporated monomer that the polymer comprises is not the same as the monomer prior to incorporation into a polymer, in that at the very least, certain linking groups are incorporated into the polymer backbone or certain groups are removed in the polymerization process, such as the loss of a water molecule in forming a peptide (amide) bond characteristic of the reaction of an amino group with a carboxyl group during PNA synthesis. A polymer is said to comprise a specific type of linkage if that linkage is present in the polymer. An incorporated monomer is a “residue”. A typical monomer for a nucleic acid or nucleic acid analog is referred to as a nucleotide or a nucleotide residue when incorporated into a polymer.

A “moiety” (pl. “moieties”) is a part of a chemical compound, and comprises groups, such as functional groups. As such, a nucleobase moiety is a nucleobase that is modified by attachment to another compound moiety, such as a polymer monomer, e.g. the nucleic acid or nucleic acid analog monomers described herein, or a polymer, such as a nucleic acid or nucleic acid analog as described herein. In chemical structures provided herein, wavy lines indicate the location of a bond linking the depicted moiety or group to a remainder of a described compound or molecule.

“Alkyl” refers to straight, branched chain, or cyclic hydrocarbon groups including from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of straight alkyl groups include methyl, ethyl, propyl, butyl, pentyl, hexyl, heptyl, octyl, nonyl, and decyl. Branched alkyl groups comprises any straight alkyl group substituted with any number of alkyl groups. Non-limiting examples of branched alkyl groups include isopropyl, isobutyl, sec-butyl, and t-butyl. Non-limiting examples of cyclic alkyl groups include cyclopropyl, cyclobutyl, cyclopentyl, cyclohexyl, cycloheptlyl, and cyclooctyl groups. Cyclic alkyl groups also comprise fused-, bridged-, and spiro-bicycles and higher fused-, bridged-, and spiro-systems. A cyclic alkyl group can be substituted with any number of straight, branched, or cyclic alkyl groups. “Alkylene” and “substituted alkylene” refer to divalent alkyl and divalent substituted alkyl, respectively, including, without limitation, ethylene (—CH₂—CH₂—).

“Aryl,” alone or in combination refers to an aromatic ring system such as phenyl, benxyl, or naphthyl. “Aryl” also includes aromatic ring systems that are optionally fused with a cycloalkyl ring. A “substituted aryl” is an aryl that is independently substituted with one or more substituents attached at any available atom to produce a stable compound. Common substituents include, but are not limited to halide atoms, such as Cl, Br, and F. “Optionally substituted aryl” refers to aryl or substituted aryl. “Arylene” denotes divalent aryl, and “substituted arylene” refers to divalent substituted aryl. “Optionally substituted arylene” refers to arylene or substituted arylene. As used herein, the term “polycyclic aryl group” and related terms, such as “polycyclic aromatic group” means a group composed of at least two fused aromatic rings. “Heteroaryl” or “hetero-substituted aryl” refers to an aryl group substituted with one or more heteroatoms, such as N, O, P, and/or S. Arylalkyl refers to moieties comprising alkyl and aryl constituents.

“Carboxyl” or “carboxylic” refers to group having the indicated number of carbon atoms and terminating in a —C(O)OH group, thus having the structure -R—C(O)OH, where R is a divalent organic group that includes linear, branched, or cyclic hydrocarbons. Non-limiting examples of these include: C₁₋₈ carboxylic groups, such as ethanoic, propanoic, 2-methylpropanoic, butanoic, 2,2-dimethylpropanoic, pentanoic, etc.

A conformationally preorganized nucleic acid analog is a nucleic acid analog that has a backbone (a preorganized backbone) that forms only either a right-handed helix or a left-handed helix, depending on the structure of the nucleic acid backbone. As shown herein, an example of a conformationally preorganized nucleic acid analog is γPNA, which has a chiral center at the γ carbon, and, depending on, and due to, the chirality of the groups at the γ carbon, forms a right-handed helix or a left-handed helix.

In the context of the present disclosure, a PNA monomer, e.g., a γPNA monomer, refers to a monomer comprising at least one nucleobase and a backbone element (backbone moiety), which in a peptide nucleic acid is N-(2-aminoethyl)-glycine, which in γPNA has a chiral center at the gamma carbon. PNA monomers also comprise reactive amine and carboxyl groups that permit polymerization under specific conditions, as in SPPS. For chemical synthesis of PNAs, the nucleobases and backbone monomers may contain modified groups, such as blocked amines, as are known in the art. A “PNA monomer residue” refers to a single PNA monomer that is incorporated into a PNA oligomer, with adjacent PNA monomers being linked by an amide bond. A “genetic recognition reagent”, in context of the present disclosure, refers generically to a peptide nucleic acid that comprises a sequence of nucleobases that is able to hybridize to a complementary nucleic acid or nucleic acid analog sequence on a nucleic acid by cooperative base pairing, e.g., Watson-Crick base pairing or Watson-Crick-like base pairing.

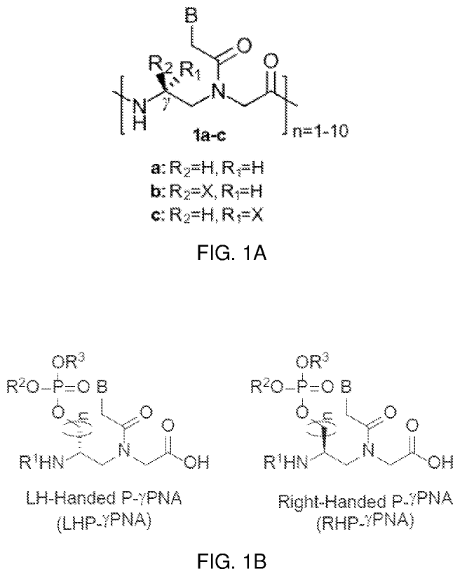

In reference to FIG. 1A, a is PNA (achiral), b is right-handed γPNA, and c is left-handed γPNA. In typical γPNA, X may be any compatible group, such as, for example and without limitation:
(1) Amino acid sidechains (Ala, CH₃; Val, CH(CH₃)₂; Ile, CH(CH₃)CH₂CH₃; Leu, CH₂CH(CH₃)₂; Met, CH₂CH₂SCH₃; Phe, CH₂C₆H₅; Tyr, CH₂C₆H₄OH; Trp, CH₂C₈I—₁₅NH; Ser, CH₂OH; HSer, CH₂CH₂OH; Thr, CHCH₃OH; Asn, CH₂CONH₂; Gin, CH₂CH₂CONH₂; Cys, CHSH; Sec, CH₂SeH; Gly, H; Pro, —(CH₂)₃—; Arg, (CH₂)₃NHC(NH)NH₂; His, CH₂C₃H₃N₂; Lys, (CH₂)₄NH₂; Asp, CH₂CO₂H; and Glu, (CH₂)₂CO₂H).
(2) Linear or branched (C₁-C₈)alkyl, (C₂-C₈)alkenyl, (C₂-C₈)alkynyl, (C₃-C₈)aryl, (C₃-C₈)cycloalkyl, (C₃-C₈)aryl(C₁-C₆)alkylene, (C₃-C₈)cycloalkyl(C₁-C₆)alkylene, —CH₂—(OCH₂—CH₂)_{q}OH, —CH₂—(OCH₂—CH₂)_{q}—NH₂, —CH₂—(OCH₂—CH₂)_{q}—NHC(NH)NH₂, —CH₂—(OCH₂—CH₂—O)_{q}—SH and —CH₂—(SCH₂—CH₂)_{q}—SH, —(CH₂CH₂)_{q}—NHC(NH)NH₂, where subscript q is an integer between 0-25.

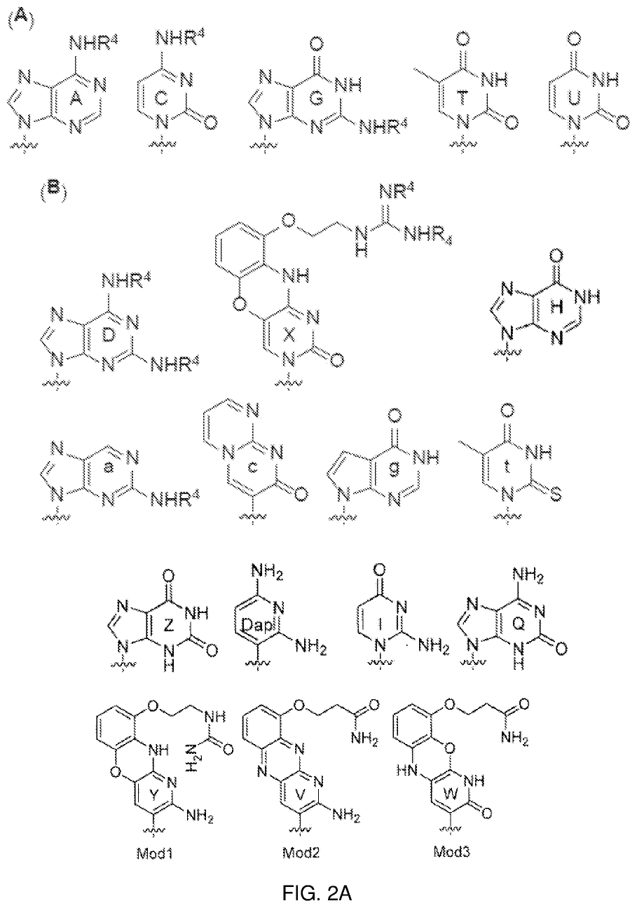

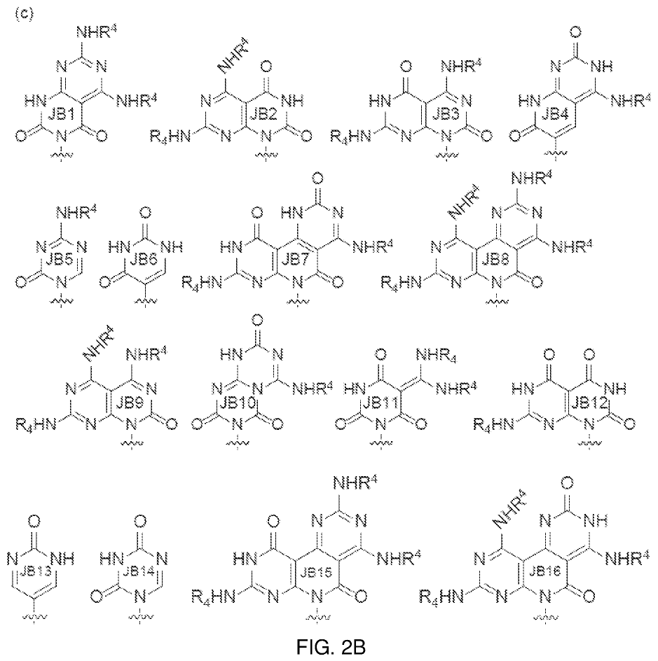

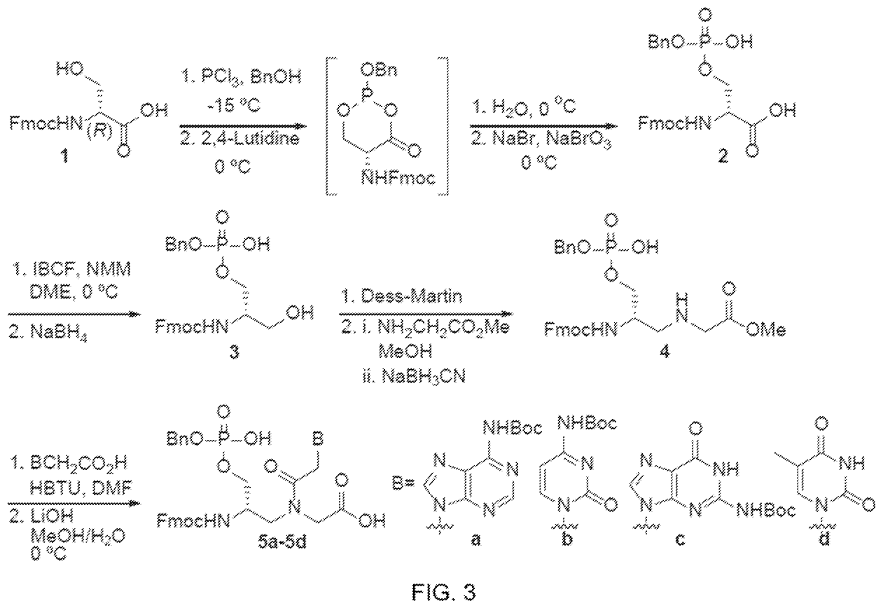

Referring to FIGS. 1A and 1B, each instance of B comprises a nucleobase, such as any one of the nucleobases (both natural and unnatural pairs and the derivatives therefor) depicted in FIGS. 2A and 2B. One or more of the nucleobases, independently, may be orthogonal nucleobases (see also, e.g., International Patent Publication Nos. WO 2014/169206, WO 2018/058091, WO 2019/126638, and WO 2019/236979, the disclosure of each of which is incorporated herein by reference, for their description of the described, additional nucleobases).

According to one aspect or embodiment, a PNA monomer synthesis method is provided that may be conducted as a one-pot reaction to install a phosphate group on amine-protected serine or homoserine, e.g., Fmoc-serine or Fmoc-homoserine (see, e.g., compound 2, FIG. 3), without the need for carboxyl group protection. Referring the FIG. 3, an exemplary scheme is provided for synthesis of phosphorylated γPNA (PγPNA). FIG. 3, though depicting a synthesis scheme for LγPNA, it is understood that the reactions are equally applicable to RyPNA, and precursors and intermediates of the same chirality, as well as racemic mixtures. As shown in FIG. 3, the synthesis method may include the conversion of alcohol (compound 3) to monomers (compounds 5a-d), as depicted.

A benzyl (Bn) protecting group may be used to protect the phosphate due to its stability toward both acid and base conditions employed in the synthesis, and that it can be simultaneously removed in a final cleavage step of solid-phase peptide synthesis (SPPS). A single, rather than double, Bn-protection may be used to achieve efficient monomer coupling, while providing complete protection for a phosphate group. FIG. 3 depicts use of a single Bn group to protect the phosphate group of the monomer. It is understood that the use of Fmoc (fluorenylmethoxycarbonyl) and Boc (tert-butyloxycarbonyl) protecting groups are exemplary, and are compatible with common Fmoc/Boc (tBu or Bhoc (benzhydryloxycarbonyl) protective groups may be substituted for Boc) solid phase peptide synthesis (SPSS) methods. Different orthogonal protecting groups may be selected for use in any method described herein, so long as they do not interfere with the general reaction schemes.

For the schemes shown in FIGS. 3, 5, and 6: Bn is benzyl; FmocOSu is FMOC-Succinimide; IBCCL is isobutyl chloroformate; Et is ethyl; DIEA is N,N-Diisopropylethylamine; DMF is N,N-dimethylformamide; NMM is N-Methylmorpholine; DME is 1,2-dimethoxy ethane; DMP is Dess—Martin periodinane; DCM is dichloromethane; MeGly is methylglycinate; DIEA is N,N-Diisopropylethylamine; DMT is 4,4′-dimethoxytrityl; HBTU is N,N,N′,N′-Tetramethyl-O-(1H-benzotriazol-1-yl)uronium hexafluorophosphate; TFA is 2,2,2-Trifluoroacetic acid; and ACN is acetonitrile.

In chemical synthesis schemes and methods described herein, the chemical structures of expected intermediate compounds are provided as illustrative of a major, intended product of a stated reaction, and certain illustrative reaction conditions, solvents, co-factors, catalysts, initiators, and other reactants are described. Choices of solvents, and other compounds and compositions in any give reaction mixture, as well as their concentration in the reaction mixture, and physical reaction conditions may be varied and optimized by one of ordinary skill in the art based on the present disclosure and the desired reaction outcome.

More generally, according to aspects or embodiments of the present disclosure, and as exemplified by the scheme of FIG. 3, a method of making a peptide nucleic acid monomer is provided. The method comprises: first, phosphorylating compound 1:

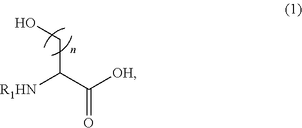

where n is 1, 2, 3, or 4, and R₁ is an amine-protecting group, e.g., with trichlorophosphorus (PCl₃), phosphoramidous acid, N,N-bis(1-methylethyl)-, bis(phenylmethyl) ester (e.g., CAS Number: 108549-23-1), or 4,3-Benzodioxaphosphepin, 3-chloro-1,5-dihydro-, 3-oxide (e.g., CAS Number: 49785-01-5), R₂—OH, and R₃—OH, to produce compound 2:

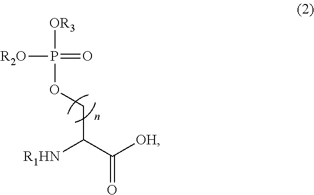

where R₂ and R₃ are, independently, H, benzyl

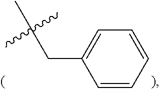 [SMILES: *Cc1ccccc1]

t-butyl, propionitrilyl

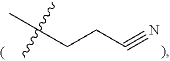 [SMILES: *CCC#N]

or 4-nitrophenylethylenyl

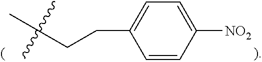 [SMILES: *CCc1ccc([N+](=O)[O-])cc1]

Compount 2 may be then reduced to produce compound 3:

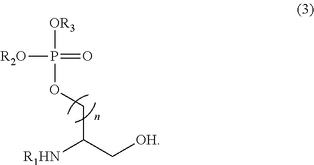

Compound 3 may be then reacted with Dess-Martin periodinane, followed by treating with NH₂CH₂C(O)OCH₃ and DCM, to produce compound 4. Alternatively compound 3 may be reacted with DMSO:TEA followed by treating with NH₂CH₂C(O)OCH₃, to produce compound 4:

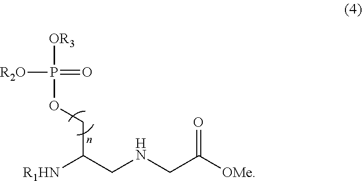

Compound 4 may then be conjugated with a nucleobase by reacting compound 4 with RCH₂C(O)OH, where R is a nucleobase in which α-amines of R are protected with an amine-protecting group, such as Boc, to produce compound 5:

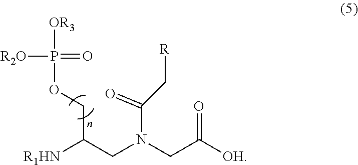

Conjugation of compound 4 may be performed using HBTU. Conjugation of a nucleobase to a PNA backbone, e.g. reacting a secondary amide of a PNA precursor with RCH₂C(O)OH, where R is a nucleobase, according to any aspect or embodiment described herein, also may be performed in the presence of alternatives to HBTU, such as, for example and without limitation: HATU (e.g., 1-[Bis(dimethylamino)methylene]-1H-1,2,3-triazolo[4,5-b]pyridinium 3-oxide hexafluorophosphate), HCTU (e.g., O-(1H-6-Chlorobenzotriazole-1-yl)-1,1,3,3-tetramethyluronium hexafluorophosphate), TATU (e.g., O-(7-azabenzotriazole-1-yl)-N,N,N′,N′-tetramethyluronium tetrafluoroborate), TBTU (e.g., 2-(1H-Benzotriazole-1-yl)-1,1,3,3-tetramethylaminium tetrafluoroborate), BOP (e.g., benzotriazol-1-yloxytris(dimethylamino)phosphonium hexafluorophosphate), PyBOP (e.g., Benzotriazole-1-yl-oxy-tris-pyrrolidino-phosphonium hexafluorophosphate), PyAOP (e.g., tripyrrolidin-1-yl(triazolo[4,5-b]pyridin-3-yloxy)phosphanium; hexafluorophosphate), PyBrOP (e.g., bromo(tripyrrolidin-1-yl)phosphanium; hexafluorophosphate), BOP-Cl (e.g., Phosphoric acid bis(2-oxooxazolidide) chloride), DCC (e.g., N,N′-dicyclohexylcarbodiimide), DIC (e.g., N,N′-Diisopropylcarbodiimide), or EDC HCl (e.g., N-(3-Dimethylaminopropyl)-N′-ethylcarbodiimide hydrochloride).

Compound 1 may be:

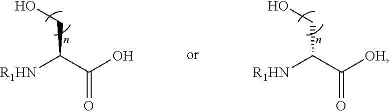

resulting in a product, compound 5 of:

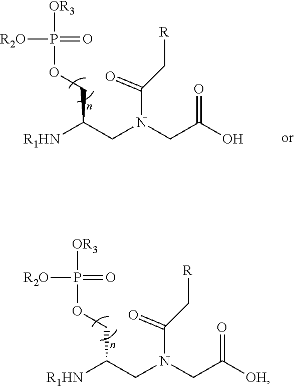

respectively.
R₁ may be Fmoc. The nucleobase R may be any nucleobase, either natural (adenine, thymine, guanine, cytosine, or uracil), or non-natural, e.g. as depicted in FIGS. 2A and 2B. In one example, R₂ and/or R₃ are benzyl.

Protecting groups (e.g., for protecting amines during synthesis of compounds described herein) are broadly-known in the art and include, for example and without limitation: 9-fluorenylmethyloxy carbonyl (Fmoc), t-butyloxycarbonyl (Boc), tert-butyl (tBu), benzhydryloxycarbonyl (Bhoc), benzyloxycarbonyl (Cbz), O-nitroveratryloxycarbonyl (Nvoc), benzyl (Bn), allyloxycarbonyl (alloc), trityl (Trt), dimethoxytrityl (DMT), I-(4,4-dimethyl-2,6-dioxacyclohexylidene)ethyl (Dde), diathiasuccinoyl (Dts), benzothiazole-2-sulfonyl (Bts) and monomethoxytrityl (MMT) groups.

PNA oligomers, e.g. comprising from 2 to 25 PNA monomer residues, as described herein, may be synthesized using solid-phase peptide synthesis (SPPS) methods, as are broadly-known, and using manual or automated equipment, including microwave-assisted methods and devices. SPPS involves the successive addition of protected amino acid derivatives, such as PNA monomers, to a growing peptide chain immobilized on a solid phase, including deprotection and washing steps to remove unreacted groups and also side products. While the SPPS methods of making PNA oligomers described herein rely on specialized reagents, such as novel monomers as described herein, the overall deprotection, conjugation, capping, and washing steps, and choice of protecting groups, may be accomplished using standard methodologies, modified as described using unique reagents and steps. Fmoc SPPS is very common, and uses Fmoc to protect terminal amines, removal of which (deprotection) permits addition of a single Fmoc-protected PNA monomer. While Fmoc SPPS is depicted throughout this document, other SPPS methods are contemplated, with appropriate choice of protective groups.

PNA oligomers, including conformationally organized RHγPNA and/or LHγPNA oligomers, have two or more PNA monomer residues, such as 2, 3, 4, 5, 6, 7, 8, 9, 10, 11, 12, 13, 14, 15, 16, 17, 18, 19, 20, 21, 22, 23, 24, or 25 consecutive PNA residues. Individual monomer residue of the PNA oligomer may be linked to (covalently attached to) a nucleobase, as depicted herein. Each individual monomer of a PNA oligomer may be linked to the same or a different nucleobase to form a defined sequence of nucleobases that may be complementary to, or bind to a nucleic acid or a nucleic acid analog, including other PNAs, as is broadly-known in the nucleic acid and peptide nucleic acid fields (see, e.g., International Patent Publication Nos. WO 2014/169206, WO 2018/058091, WO 2019/126638, and WO 2019/236979, the disclosure of each of which is incorporated herein by reference, also providing useful PNA oligomers). In a PNA oligomer, the sequence of nucleobases, and the sequence of PNA monomers, as described herein is produced by the stepwise addition of individual PNA monomers by SPSS protocols.

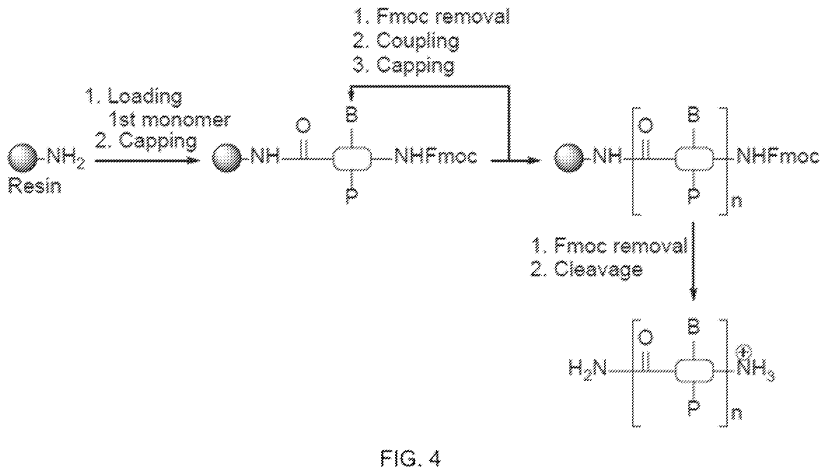

FIG. 4 depicts an exemplary Fmoc-based SPPS method using PγPNA monomers as described herein. Briefly, a first monomer is loaded onto the resin, linking the first monomer to the resin. The resin is capped, e.g., by standard capping protocols, for example and without limitation using acetic anhydride in N,N-dimethylformamide (DMF) with pyridine or N,N-diisopropylethylamine (DIPEA). Subsequent monomers may be added by sequential Fmoc removal (deprotection), coupling , and capping. Once the PNA oligomer is completed, the terminal Fmoc is removed and the peptide is cleaved from the resin. The oligomer may include one or more, or all PγPNA residues, with other compatible PNA or γPNA residues for the remainder of the oligomer. Nucleobase B for each monomer may be the same or different, and typically are added, and therefore included in the oligomer, in a specific sequence. Nucleobases may be natural or non-natural including mixtures thereof. Phosphate groups in the oligomer may include protective groups, such as benzyl groups as described herein. The protective groups for the phosphate may be removed during, prior to, or after cleavage of the PNA oligomer from the solid support, for example Bn group(s) on a phosphate moiety are removed during cleavage of the oligomer from the solid support.

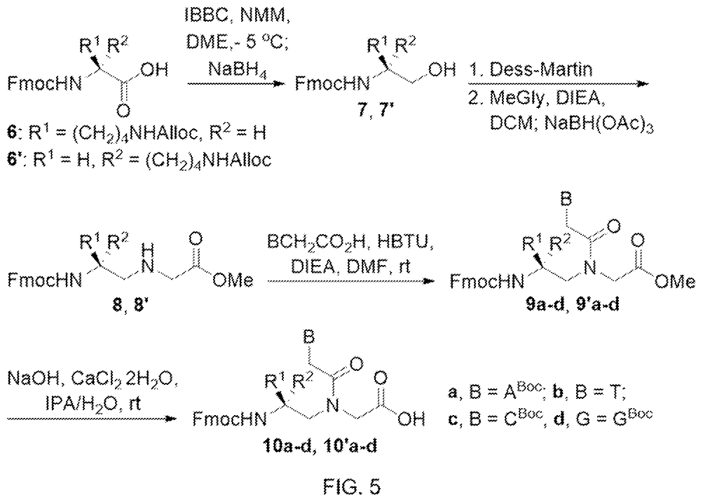

According to one aspect or embodiment, a PNA monomer synthesis method is provided that may be conducted as a one-pot reaction to install a protective allyloxycarbonyl group (—C(O)OCH₂CHCH₂ or Alloc) at the gamma carbon (see, e.g., compounds 10a-d and 10′a-d, FIG. 5). Removal of the Alloc group yields an alkyl side chain with a terminal amine, e.g., —(CH₂)ₙNH₂, where n ranges from 1 to 6, such as a lysine side chain, where n is 4. The amine of the alkyl side chain can be modified with a suitable amine-reactive compound, such as a carboxylated or triflated (Tf) compounds, for example and without limitation:

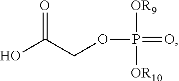

where R₉ and R₁₀ are, independently, H, benzyl, t-butyl, propionitrilyl, or 4-nitrophenylethylenyl;

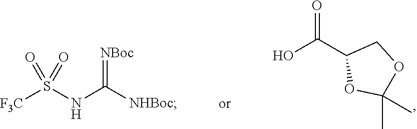

to add phosphate, guanidine, or dihydroxypropyl functionality, respectively.

According to aspects or embodiments of the present disclosure, a method of making a peptide nucleic acid monomer, as exemplified in FIG. 5, is provided, comprising: first, reducing compound 6:

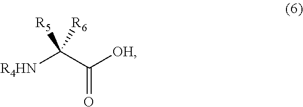

where R₄ is an amine-protecting group, and one of R₅ and R₆ is H, and the other of R₅ and R₆ is:

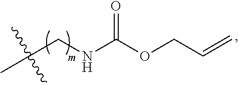

where m is 1, 2, 3, or 4 (methylenyl, dimethylenyl, trimethylenyl, or tetramethylenyl), to produce compound 7:

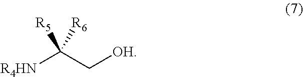

It is noted that the location of the H atom for R₅ and R₆ will determine chirality of the resultant monomer. Next compound 7 may be reacted with Dess-Martin periodinane, followed by treating with NH₂CH₂C(O)OCH₃ (methyl glycinate), or reacted with DMSO:TEA followed by treating with methyl glycinate, to produce compound 8:

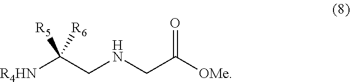

Compound 8 may then be conjugated to a nucleobase at the secondary amine by linking with RCH₂C(O)OH, where R is a nucleobase in which α-amines of R are protected with an amine-protecting group to produce compound 9:

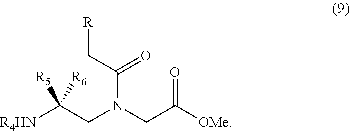

The terminal methyl group of 9 is then removed to produce compound 10:

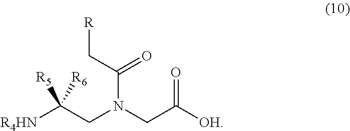

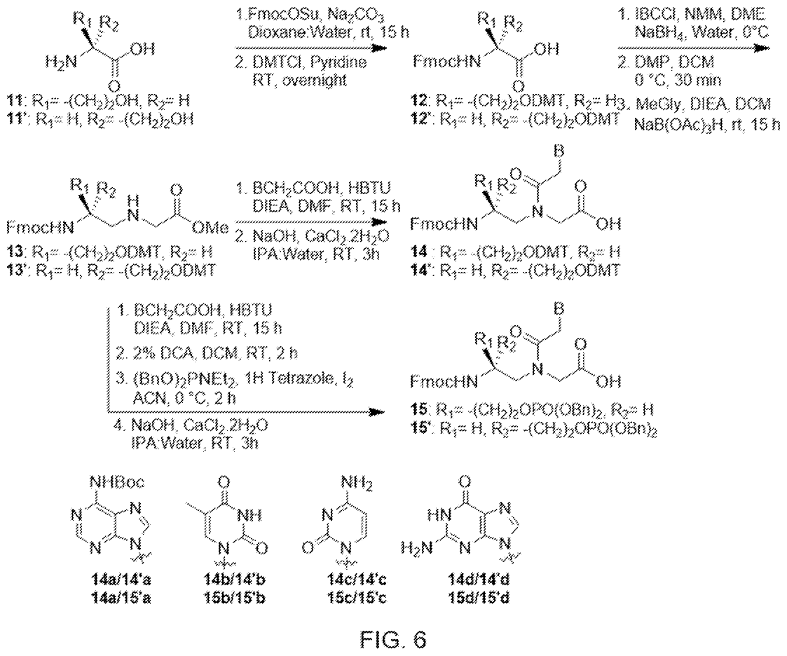

According to one aspect or embodiment, a PNA monomer synthesis method is provided that may be conducted as a one-pot reaction to install a phosphate at the gamma carbon (see, e.g., compounds 15a-d and 15′a-d, FIG. 6). FIG. 6 provides a scheme for producing compounds 15a-d and 15′a-d. of any chirality, and with any useful nucleobase as exemplified by a-d. This method exploits the ability of DMT to participate in phosphorylation reactions.

In one aspect or embodiment, a method of making a peptide nucleic acid monomer is provided. The method comprises, first, adding an amine protecting group to the terminal amine and adding a 4,4′-dimethoxytrityl (DMT) group to the primary hydroxyl group of compound 11:

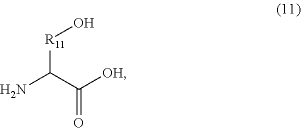

where R₁₁ is —(CH₂)ₙ—, and n is 1-4, to produce compound 12;

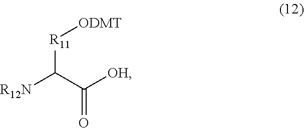

where R₁₂ is an amine-protecting group, such as Fmoc. Compound 12 may then be reduced, and reacted with Dess-Martin periodinane and methyl glycinate to produce compound 13:

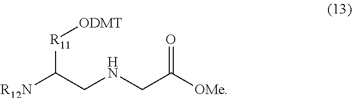

Compound 13 may next be conjugated with a nucleobase by reacting compound 13 with RCH₂C(O)OH, where R is a nucleobase in which α-amines of R are protected with an amine-protecting group, and the methyl group may be removed from the terminal carboxymethyl group, to produce compound 14:

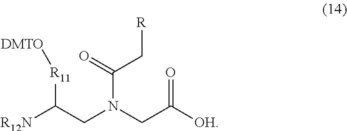

The DMT-protected oxygen of compound 14 may then be phosphorylated with dichloroacetic acid,

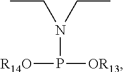

tetrazole, and I₂, to produce compound 15:

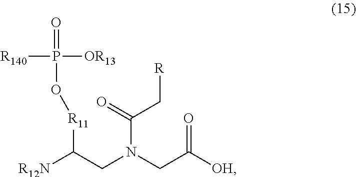

where R₁₃ and R₁₄ may be, independently, H, benzyl, t-butyl, propionitrilyl, or 4-nitrophenylethylenyl.

It is noted that in all phosphorylated γPNA monomers described herein, one or more of the oxygen atoms of the phosphate group may be connected to benzyl, t-butyl, propionitrilyl, or 4-nitrophenylethylenyl groups. These groups may serve to protect the phosphate group and may be removed during cleavage of the PNA oligopeptide from the solid support during SPSS incorporation of the PNA monomer.

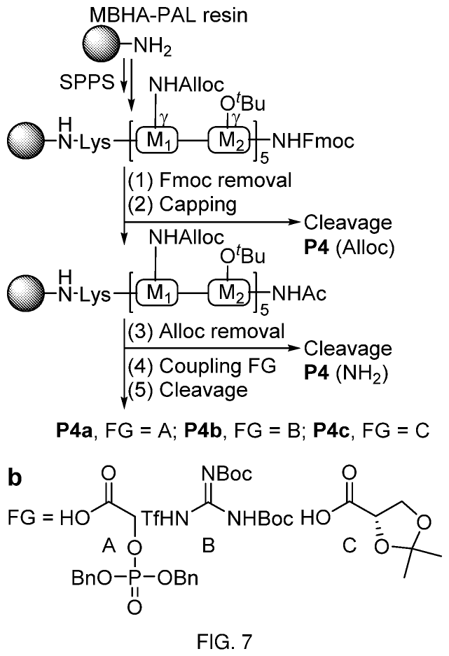

PNA monomers synthesized as described above, for example as shown in FIGS. 5 and 6, having Alloc or DMT groups protecting the alkyl group pending from the gamma carbon may be used in a SPSS method to produce γPNA oligomers. FIG. 7 depicts an Fmoc-based SPSS (Fmoc SPSS) synthesis scheme for PNA oligomers comprising Alloc-modified lysinyl side chains at the chiral gamma carbon of one or more PNA monomer residues, e.g., as described above. Oligomers comprising residues of PNA monomers with DMT-modified side chains at the chiral gamma carbon of one or more PNA monomer residues, e.g., as described above, may be similarly processed as depicted in FIG. 7, however, the DMT groups may be replaced by phosphate as described above, as exemplified in FIG. 6, before incorporation of the monomer into a PNA oligomer, essentially as depicted as described above, as exemplified in FIG. 4. It would be appreciated that not all PNA monomer residues of a synthesized PNA oligomer would comprise the same groups at the gamma carbon or elsewhere on their backbones, or have the same nucleobases. It also would be appreciated that the depicted method in FIG. 7 is equally applicable to RH-γPNA monomers and oligomers. As shown in Example 4, PNAs were synthesized with different numbers of PNA monomers with gamma carbons modified as described herein. In reference to FIG. 7, the Alloc groups may be removed, deprotecting the amine before or after cleavage of the PNA oligomer from the resin. Likewise, groups reactive with the deprotected amine may be linked to that amine before or after cleavage of the PNA oligomer from the resin. Where phosphate is linked to the gamma carbon, one or more oxygens of the phosphate may be protected, e.g. with a Bn protective group, in which case cleavage of the peptide from a support or resin removes the Bn protective group.

In the case of the process exemplified in FIG. 5, the Alloc group is removed, e.g., using Pd(PPh₃)₄ (palladium-tetrakis(triphenylphosphine)) or PhSiH₃ (phenylsilane), and the remaining amine group is linked to an amine-reactive group, such as a phosphate, a guanidine, a dihydroxypropyl group, or a dye. Amine-reactive groups may be provided in the form of:

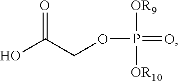

where R₉ and R₁₀ are, independently, H, benzyl, t-butyl, propionitrilyl, or 4-nitrophenylethylenyl;

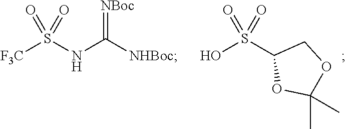

glyceric acid; dyes, such as cyanine, FAM, FITC, Rhodamine dyes; and R₁₅—C(O)OH (e.g., organic acids or carboxylic acids), where R₁₅ may be an alky or aryl group, including alkyl-aryl, or substituted alkyl, aryl, or alkyl-aryl groups.

In the case of the process exemplified in FIG. 6, the -ODMT group is replaced by a phosphate, e.g., a protected phosphate.

The methods described herein may be used to produce a genetic recognition reagent, that binds specifically to fully or partially complementary nucleic acid or PNA strands. The genetic recognition reagent comprises a plurality of nucleobase moieties, each attached to a PNA backbone monomer residue, and forming a part of the larger genetic recognition reagent comprising at least two PNA monomer residues, and therefore at least two nucleobases (nucleobase moieties). Depending upon choice of nucleobases in the sequence, the genetic recognition reagents described herein can bind a single nucleic acid or PNA strand, or invade or otherwise hybridize to two strands of fully-complementary, partially-complementary or non-complementary double-stranded nucleic acids. As used herein, a monovalent nucleobase binds one nucleobase on a single nucleic acid strand, while a divalent nucleobase binds to two nucleobases, one on a first nucleic acid strand, and another on a second nucleic acid strand. Any choice of divalent and/or monovalent nucleobases may be selected for incorporation into a PNA oligomer as described herein.

Complementary refers to the ability of polynucleotides (nucleic acids) to hybridize (bind) to one another, forming inter-strand base pairs. Base pairs are formed by hydrogen bonding between nucleotide units in antiparallel polynucleotide strands. Complementary polynucleotide strands can base pair (hybridize or bind) in the Watson-Crick manner (e.g., A to T, A to U, C to G), or in any other manner that allows for the formation of duplexes. When using RNA as opposed to DNA, uracil rather than thymine is the base that is complementary to adenosine. Two sequences comprising complementary sequences can hybridize if they form duplexes under specified conditions, such as in water, saline (e.g., normal saline, or 0.9% w/v saline) or phosphate-buffered saline), or under other stringency conditions, such as, for example and without limitation, 0.1×SSC (saline sodium citrate) to 10×SSC, where 1×SSC is 0.15M NaCl and 0.015M sodium citrate in water. Hybridization of complementary sequences is dictated, e.g., by salt concentration and temperature, with the melting temperature (Tm) lowering with increased mismatches and increased stringency. Perfectly matched sequences are said to be “fully complementary”, though one sequence (e.g., a target sequence in an mRNA) may be longer than the other, as in the case of the small recognition reagents described herein in relation to the much longer target sequences on which they concatenate, such as mRNAs containing repeat expansions. Two complementary strands of nucleic acid bind in an antiparallel orientation, with one strand in a 5′ to 3′ orientation, and the other in a 3′ to 5′ orientation. PNA permits both parallel and antiparallel orientation, though for γPNA antiparallel binding is preferred.

Examples of applications for the oligomers described herein is in the treatment of genetic diseases with repeat expansion of small sequences, such as those listed in Table 1.

TABLE 1
Genetic diseases associated with expanded repeats

| | | | Normal | Pathogenic |
|---|---|---|---|---|
| Disease | Repeat Unit | Gene Name | Repeat Length | Repeat Length |
| FRDA | (GAA)n | FRDA (frataxin) | 6-32 | 200-1,700 |
| FRAXA | (CGG)n | FMR1 (FMRP) | 6-60 | >200 |
| FRAXE | (CCG)n | FMR2 (FMR2) | 4-39 | 200-900 |
| SCA1 | (CAG)n | SCA1 (ataxin 1) | 6-39 | 40-82 |
| SCA2 | (CAG)n | SCA2 (ataxin 2) | 15-24 | 32-200 |
| SCA3 (MJD) | (CAG)n | SCA3 (ataxin 3) | 13-36 | 61-84 |
| SCA6 | (CAG)n | CACNA1A | 4-20 | 20-29 |
| SCA7 | (CAG)n | SCA7 (ataxin 7) | 4-35 | 37-306 |
| SCA17 | (CAG)n | SCA17 (TBP) | 25-42 | 47-63 |
| DRPLA | (CAG)n | DRPLA (atrophin 1) | 7-34 | 49-88 |
| SBMA | (CAG)n | AR (androgen | 9-36 | 38-62 |
| | | receptor) | | |
| HD | (CAG)n | HD (huntingtin) | 11-34 | 40-121 |
| MD1 | (CTG)n | DMPK (DMPK) | 5-37 | 50-1,000 |
| MD2 | (CCTG)n | ZNF9 (ZNF9) | 10-26 | 75-11,000 |
| FXTAS | (CGG)n | FMR1 (FMRP) | 6-60 | 60-200 |
| SCA8 | (CTG)n | SCA8 | 16-34 | >74 |
| SCA10 | (ATTCT)n | Unknown | 10-20 | 500-4,500 |
| SCA12 | (CAG)n | PPP2R2B | 7-45 | 55-78 |
| HDL2 | (CTG)n | JPH3 | 7-28 | 66-78 |
| ALS | (GGGGCC)n | C9ORF72 | 20-50 | >100 |

Based on Table 1, the nucleobase sequence of the PNA oligomers described herein would bind to the repeat sequences shown in that table, either to a single strand, or to two strands in the case of divalent nucleobases for genetic recognition reagents. The PNA oligomer may bind to a single repeat or contain more than one iterations of the repeated sequence. It should be noted that, depending on their sequence, not all repeated sequences will form a hairpin structure under normal conditions, but can be induced into a triplex “hairpin” structure by a genetic recognition reagent comprising divalent nucleobases. The repeated nature of the sequences dictate that for a three-base repeat, three different frameshifts may be useful for each sequence, and for a four-base repeat, four different frameshifts may be useful. In use, PNA oligopeptides as described herein may be compounded or formulated into a pharmaceutical composition, including one or more pharmaceutically-acceptable excipients.

The following are illustrative examples of the preparation of γPNA monomers and oligomers as described herein.

### EXAMPLE 1

### Production of Phosphorylated γPNA Monomer

The overall scheme for this reaction is essentially as depicted in FIG. 3. Compound 2 was prepared essentially as in Mowrey et al. Demonstration of a Scalable One-Pot Synthesis of Fmoc-O-Benzylphospho-I-serine Org. Process Res. Dev. 2012, 16, 1861-1865.

Compound 3: To a cold solution of compound 2 in anhydrous DME was added NMM followed by IBCF under argon atmosphere. After stirring the reaction mixture at the same temperature for 10 minutes, the mixture was filtered, and the residue was washed with DME. The collected filtrate was treated with an aqueous NaBH₄ solution at −10 C. The reaction mixture was poured into water. The precipitate was collected by vacuum filtration and washed with water to afford compound 3 as a white solid, which was used in the next step without further purification.

Compound 4: 6 mmol of pyridine in anhydrous CH₂Cl₂ was added to an argon-purged flask containing Dess-Martin periodinane (1 mmol) in anhydrous CH₂Cl₂ to generate a clear solution. This stock solution was used in the oxidation step within 20 min. To a solution of compound 3 (1 eq) was added (1.9 eq.) of freshly prepared periodinane stock solution in one portion. After 1 h, the clear solution was diluted with 5% MeOH:CH₂Cl₂ and was quenched by adding 1:1 saturated aqueous NaHCO₃/sodium bisulfite and stirring the resulting mixture for 5 min. The mixture was washed with saturated aqueous NaHCO₃ and brine, and the organic layer was dried (Na₂SO₄), filtered, and concentrated. The resulting oily residue was used in the next step without further purification.

To an aldehyde solution in anhydrous DCM was added glycine hydrochloride followed by DIPEA. After 1 h at room temperature, NaB(OAc)₃H was added to the reaction mixture and left for overnight stirring. The reaction mixture was diluted with 10% MeOH:DCM: 2% aq. HCl solution and the solution was extracted with 10% MeOH:DCM. The combined organic layer was dried (Na₂SO₄) and removed. The resultant crude was purified using silica gel column chromatography to obtain compound 4.

Compound 5: The mixture of nucleobase acid, HBTU, DIPEA, and compound 4 was stirred at room temperature for 10-15 hours. After consuming one of the starting materials, the reaction mixture was concentrated under a vacuum. The resultant residue was dissolved in water and extracted with 10% MeOH:DCM (2×). The combined organic layer was dried and removed. The obtained residue was purified by using silica gel column chromatography.

1N NaOH was added to the solution of methyl ester monomer and CaCl₂.XH₂O in IPA:H₂O mixture. After 3 h at room temperature, the reaction mixture was acidified with acetic acid and concentrated. The remaining residue was treated with MeOH and water. The obtained precipitate was collected by vacuum filtration and washed with water.

### EXAMPLE 2

### Use of Alloc to Produce γPNA Monomer

The overall scheme for this reaction is essentially as depicted in FIG. 5, which shows the synthesis scheme for a series of modifiable γPNA monomers with a lysine side chain at the gamma position. The synthesis of RH-gAllocPNA and LH-gAllocPNA monomers have been accomplished using commercially available Fmoc-L-Lys(Alloc)-OH and Fmoc-D-Lys(Alloc)-OH as starting materials. The reduction of the carboxylic acid of Lysine (6 & 6′) to the alcohol (7 & 7′) was carried out using known protocols. However, the chemical transformation of alcohols (7 & 7′) from the previous step to the PNA backbone is critical as it involves the racemization of a PNA backbone. Therefore, an optically pure PNA backbone (8 & 8′) was afforded from compounds 7 and 7′ using mild oxidation of alcohol and reductive amination of an aldehyde, to which all four nucleobases were coupled. Ultimately, the saponification of methyl ester yielded optically pure RH- and LH-PNA monomers

Compound 7: To a cold solution of compound 6 in anhydrous DME was added NMM followed by IBCF under argon atmosphere. After stirring the reaction mixture at the same temperature for 10 minutes, the mixture was filtered, and the residue was washed with DME. The collected filtrate was treated with an aqueous NaBH₄ solution at −5° C. The reaction mixture was poured into water. The obtained precipitate was collected by vacuum filtration and washed with water to afford compound 7 as a white solid, which was used in the next step without further purification.

Compound 8: To a cold solution of compound 7 (1 eq) was added Dess-Martin periodinane (1.15 eq.) in portion-wise and warmed to room temperature. After 1 h, the solution was diluted with ether and quenched by adding 1:1 saturated aqueous NaHCO3/sodium thiosulfate and stirring the resulting mixture for 5 minutes. The mixture was washed with saturated aqueous NaHCO₃ and brine, and the organic layer was dried (Na₂SO₄), filtered, and concentrated. The resulting residue was used in the next step without further purification.

To an aldehyde solution (1eq) in anhydrous DCM was added glycine hydrochloride (1.5eq) followed by DIPEA (2.5 eq). After 1 h at room temperature, NaB(OAc)₃H (2 eq) was added to the reaction mixture and left for overnight stirring. The reaction mixture was diluted with DCM solution, and the resulting solution was washed with 10% sodium bicarbonate solution. The combined organic layer was dried (Na₂SO₄) and removed. The resultant crude was purified using silica gel column chromatography to obtain compound 8.

Compound 9: To the mixture of nucleobase acid (1 eq), HBTU (1 eq), DIPEA (1.1 eq) in anhydrous DMF was added compound 8 (1 eq) at room temperature and stirred for an additional 10-15 hours. After consuming one of the starting materials, the reaction mixture was concentrated under a vacuum. The resultant residue was dissolved in water and extracted with ethyl acetate (2×). The combined organic layer was dried and removed. The obtained residue was purified by using silica gel column chromatography.

1N NaOH (1.5 eq) was added to the solution of Methyl ester monomer (1 eq) and CaCl₂.XH₂O (20 eq) in IPA:H₂O mixture. After 3 h at room temperature, the reaction mixture was acidified with acetic acid and concentrated. The remaining residue was treated with MeOH and water. The obtained precipitate was collected by vacuum filtration and washed with water and purified by silica gel column chromatography to obtain pure monomer series 9.

### EXAMPLE 3

### Use of DMT to Produce γPNA Monomer

The overall scheme for this reaction is essentially as depicted in FIG. 6. Compound 12 was prepared essentially as described in Filira F, et al. Opioid peptides: synthesis and biological properties of [(N gamma-glucosyl,N gamma-methoxy)-alpha, gamma-diamino-(S)-butanoyl]4-deltorphin-1-neoglycopeptide and related analogues. Org Biomol Chem. 2003 Sep. 7; 1(17):3059-63 and John Nielsen, et al. Sydney Brenner, and Kim D. Janda Synthetic methods for the implementation of encoded combinatorial chemistry Journal of the American Chemical Society 1993 115 (21), 9812-9813.

Compound 13: (i) To a cold solution of compound 2 in anhydrous DME was added NMM followed by IBCF under argon atmosphere. After stirring the reaction mixture at the same temperature for 10 minutes, the mixture was filtered, and the residue was washed with DME. The collected filtrate was treated with an aqueous NaBH₄ solution at −10 C. The reaction mixture was poured into water. The precipitate was collected by vacuum filtration and washed with water to afford compound 12 as a white solid, which was used in the next step without further purification.

(ii): 6 mmol of pyridine in anhydrous CH₂Cl₂ was added to an argon-purged flask containing Dess-Martin periodinane (1 mmol) in anhydrous CH₂Cl₂ to generate a clear solution. This stock solution was used in the oxidation step within 20 min. To a solution of step 1 compound (1 eq) was added (1.9 eq.) of freshly prepared periodinane stock solution in one portion. After 1 h, the clear solution was diluted with Ether and was quenched by adding 1:1 saturated aqueous NaHCO₃/sodium bisulfite and stirring the resulting mixture for 5 min. The mixture was washed with saturated aqueous NaHCO₃ and brine, and the organic layer was dried (Na₂SO₄), filtered, and concentrated. The resulting oily residue was used in the next step without further purification.

(iii) To an aldehyde solution in anhydrous DCM was added glycine hydrochloride followed by DIPEA. After 1 h at room temperature, NaB(OAc)₃H was added to the reaction mixture and left for overnight stirring. The reaction mixture was diluted with DCM and the solution was extracted with DCM. The combined organic layer was dried (Na₂SO₄) and removed. The resultant crude was purified using silica gel column chromatography to obtain compound 13.

Compound 14: The mixture of nucleobase acid, HBTU, DIPEA, and compound 13 was stirred at room temperature for 10-15 hours. After consuming one of the starting materials, the reaction mixture was concentrated under a vacuum. The resultant residue was dissolved in water and extracted with Ethyl acetate (2×). The combined organic layer was dried and removed. The obtained residue was purified by using silica gel column chromatography.

1N NaOH was added to the solution of methyl ester monomer and CaCl₂.XH₂O in IPA:H₂O mixture. After 3 h at room temperature, the reaction mixture was acidified with acetic acid and concentrated. The remaining residue was treated with MeOH and water. The obtained precipitate was collected by vacuum filtration and washed with water and purified by silica gel column chromatography.

### EXAMPLE 4

### Preparation of γPNA Oligomers

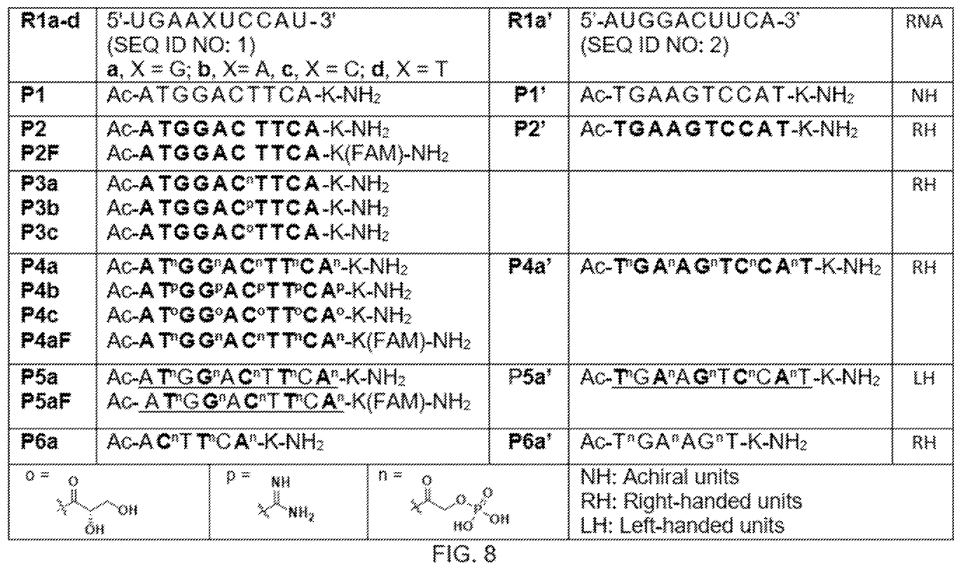

Six sets of PNA oligomers were prepared as shown in FIG. 8, using Fmoc solid-phase PNA synthesis. The PNA P1 and P2 were used as the controls for binding studies comparison. The series 3a-3c were designed to study the influence of each external modification on hybridizing these oligomers with complementary PNA or RNA R1 (Mismatched RNA, R2). In the fourth and fifth set of PNA (P4 and P5 series), the alternate charged or neutral models were designed to demonstrate the effect on PNA:PNA and PNA:RNA biophysical properties, orthogonal binding, and orthogonal toe-hold binding of PNA:PNA in the presence of complementary RNA sequences. PNA monomer residues not identified as being modified with o, p, or n, were: (1) for P1 and P1′, achiral, with H at both positions at the gamma carbon (e.g. in reference to FIG. 1A R₁ and R₂ are both H), and (2) for the chiral RH-γPNA and LH-γPNA oligomers, are serinyl, that is they comprise an H and a —CH₂—OH at the gamma carbon, arranged at the gamma carbon in the stated chirality for the oligomer indicated in FIG. 8 (e.g. in reference to the structure of FIG. 1A, X is —CH₂OH, and the —CH₂OH is arranged at R₁ or R₂ with the chirality indicated in FIG. 8).

In further detail, Fmoc based solid-phase synthesis was used to to prepare all PNA oligomers depicted in FIG. 8. After complete sequence synthesis, the N-terminus amine of each oligomer was capped with acetic anhydride to avoid ambiguity between each lysine's epsilon amine and the N-terminus amine. The alloc moiety was selectively deprotected in the presence of O-tBu and Boc protecting group using palladium as a catalyst and phenyl silane as a scavenger to generate orthogonal amine. The resultant amine on the resin support was successfully coupled to respective groups to obtain charged and neutral oligomers from the same oligomer as a starting material. Di-benzyl protection group on phosphate's hydroxyl moiety was chosen to allow easily removal of the protection group and stability throughout the synthesis to generate a negatively charged oligomer. Guanidine triflate and protected glyceric acid were used to produce positively- and neutrally-charged oligomers, respectively.

### EXAMPLE 5

### Evaluation of γPNA Oligomers

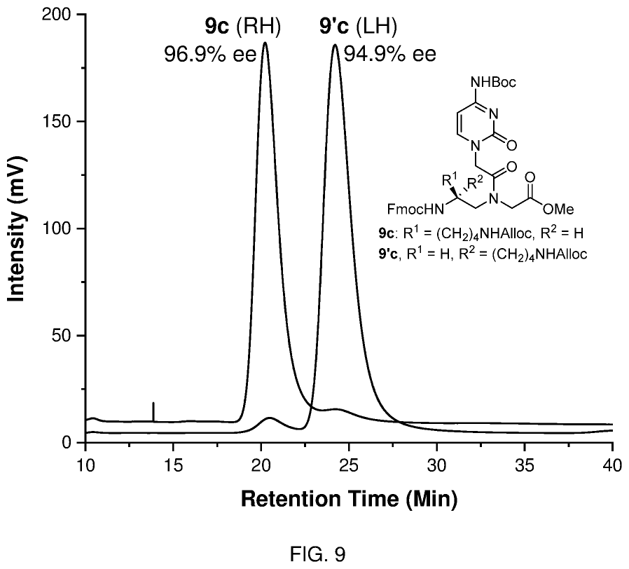

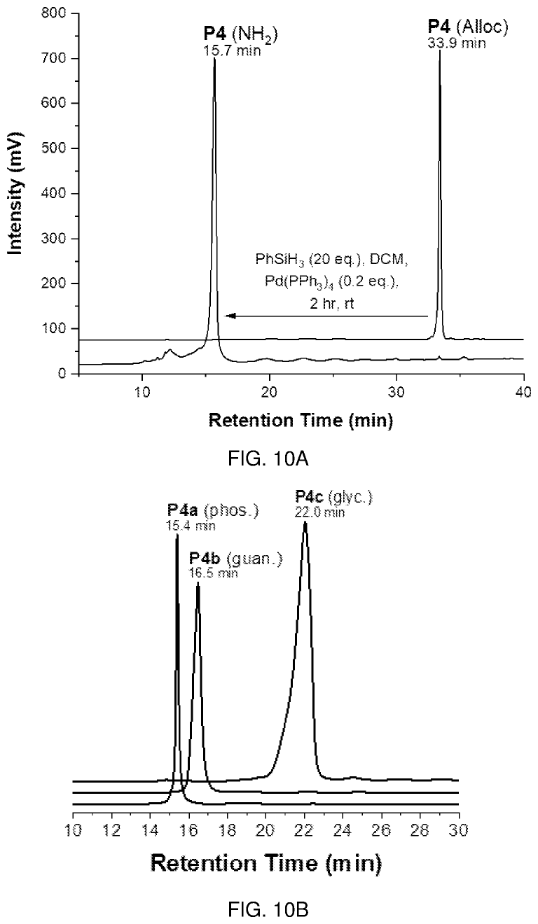

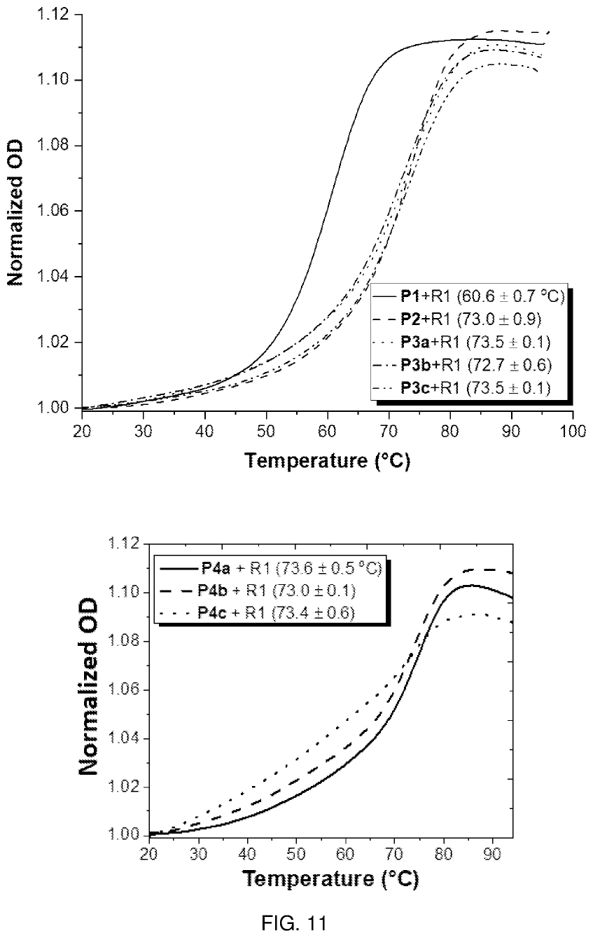

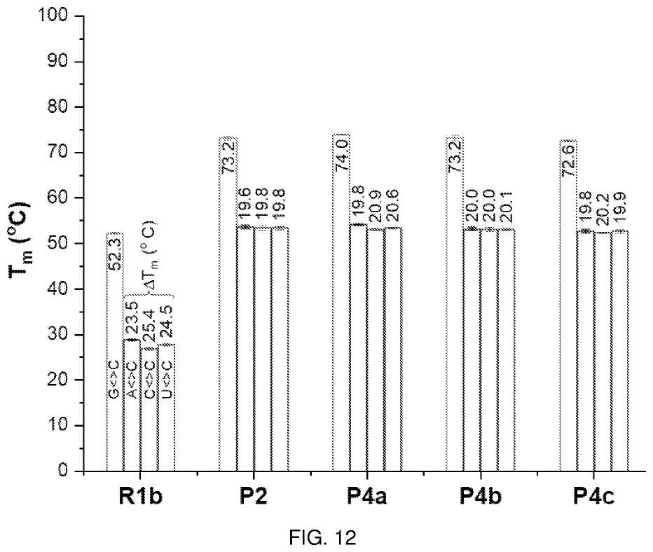

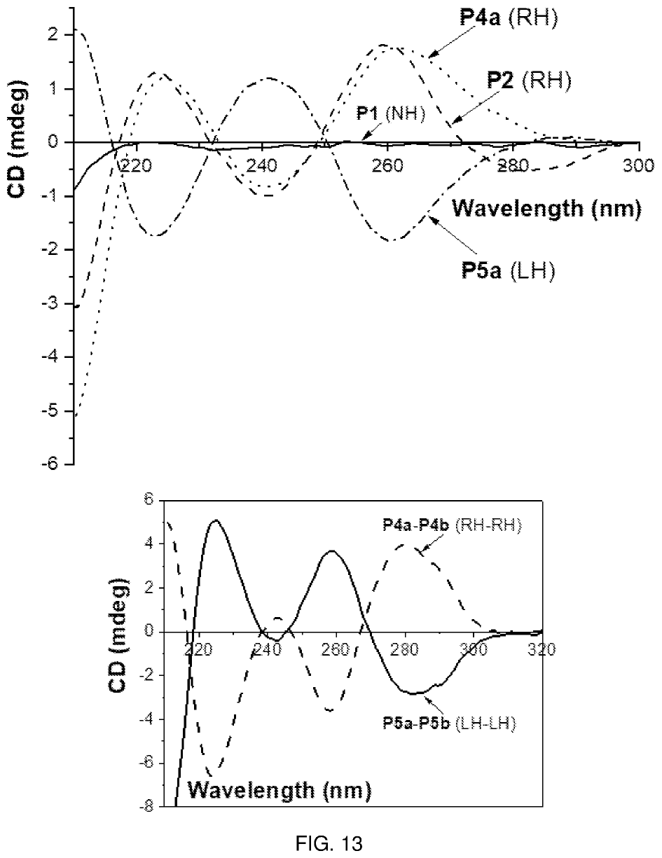

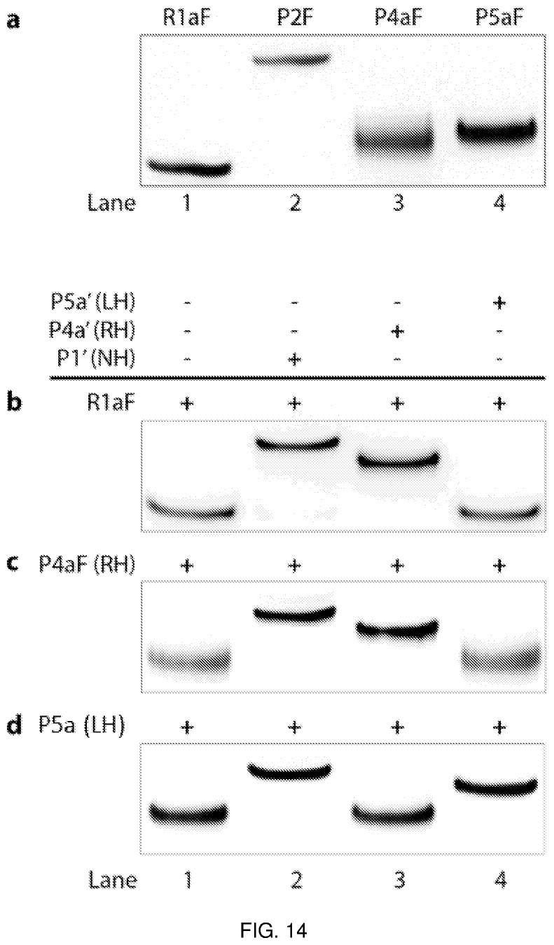

For Example 5, all assays were conducted using standard protocols. Selected PNA monomers, and the oligomers of FIG. 8 were evaluated as shown in FIGS. 9-14. FIG. 9 provides a graph showing HPLC analysis of left- and right-handed Alloc-modified γPNA monomers prepared as described, indicating high purity yields from the described synthesis methods. FIGS. 10A and 10B provide HPLC graphs showing, in the context of P4 described in FIG. 5, removal of the Alloc group (FIG. 10A), and (FIG. 10B), monomers P4A, P4b, and P4c, with differently modified groups at the gamma carbon. FIG. 11 depicts melting of the described oligomers with an RNA template. FIG. 12 shows the effects of sequence mismatch of gamma-PNA and RNA duplexes as shown, with mismatched being in the base X of R1, shown in FIG. 8. FIG. 13 provides circular dichroism (CD) profiles of the individual PNA (P1) and gammaPNA strands (P2, P4a, and P5a), along with the corresponding gammaPNA-gammaPNA duplexes. FIG. 14 depicts gel-shift assays demonstrating recognition orthogonality.

From the HPLC, the conversion of five alloc to five amines in the P4 series was very efficient. The polarity of alloc PNA was least followed by glyceric acid oligomer P4c. The polarity of P4a, P4b, and P4 (NH₂) was almost identical on HPLC.

In further detail, to validate that the post-modification of PNA oligomers will not influence the PNA-PNA or PNA-RNA thermal stability; we measured the UV-melting profiles. The thermal stability of P2:R1 was higher than P1:R1 due to the preorganized helical structure of P2. The UV-melting profiles of singly modified oligomer (P3 series) with a complementary RNA R1 showed a similar melting transition as the P2-R1 duplex. The presence of either modification on the PNA did not disturb the thermal stability of the duplex. Moreover, the five alternate alterations with different charges in parent PNA P2 (P4 series) also presented similar melting temperatures. No electrostatic interactions between gamma epsilon guanidine (P4b) or repulsion between gamma epsilon phosphate (P4a) and phosphate (R1) groups were noted.

Referring to FIG. 13, to determine the effect of external modification on the confirmation of PNA, we measured the helicity of PNA using a CD spectrometer. The CD spectra of PNA P4a reveal similar distinct exciton coupling patterns as PNA P2, with minima around 240 nm and maxima around 225 and 260 nm; in contrast to PNA P4a, PNA P5a, which showed exact inverted patterns due to preorganized left-handed helicity. The amplitude of each strand (P2 and P4a, inverted in the case of PNA P5a) remained similar. However, in the case of P4a, the minima at 290 significantly decreased and broadened the amplitude around 260 nm compared to PNA P2. The redshift appeared in P4a/P5a approximately by 4 nm. The PNA/PNA (FIG. 13, bottom) and PNA/RNA (Data not shown) duplex showed similar CD spectra except for the increased amplitude in the signal. No adverse effects were observed on the helicity of either single strand PNA or PNA-PNA and PNA-RNA duplex due to post modification on the PNA.

To determine the mobility and orthogonal binding of prepared negatively charged PNA, EMSA comparing the orthogonal binding of Right-handed (P4a) and Left-handed (P5a) with complementary RNA R1 at physiologically relevant conditions was performed. FIG. 14 depicts the mobility of PNA. P4a and P5a had ten negative charges from five phosphate moieties like RNA R1. The most retarded band (Lane 2) from the FIG. 14 (a) was for the gamma serine PNA P2. However, PNA P4a (Lane 3) and P5a (Lane 4) had approximately the same mobility as RNA 1 (Lane 1) on the EMSA. The smear band in P4a was due to two isomers of FAM dye (6-carboxyfluorescein). In the case of P5a and P2, a single isomer of FAM dye PNA was isolated. Upon confirmation of mobility of the PNA, the orthogonal binding of PNA against the complementary strand of RNA and PNA was studied. P4a formed an individual complex with RNA R1, PNA P1, and P4ap. In contrast, P5a was unable to bind to R1, and P4ap but showed retarded shift with the complete disappearance of P5aF in the presence of achiral P1 and left-handed (LH) P5ap.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of making a peptide nucleic acid monomer, comprising:
phosphorylating compound 1, where n is 1, 2, 3, or 4, R₁ is an amine-protecting group: 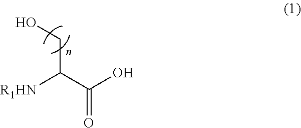
with trichlorophosphorus (PCl₃), phosphoramidous acid, N,N-bis(1-methylethyl)-, bis(phenylmethyl) ester, or 4,3-Benzodioxaphosphepin, 3-chloro-1,5-dihydro-, 3-oxide), R₂—OH, and R₃—OH, to produce compound 2, where R₂ and R₃ are, independently, H benzyl 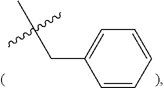 [SMILES: *Cc1ccccc1]
t-butyl, propionitrilyl 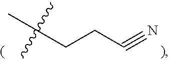 [SMILES: *CCC#N]
or 4-nitrophenylethylenyl 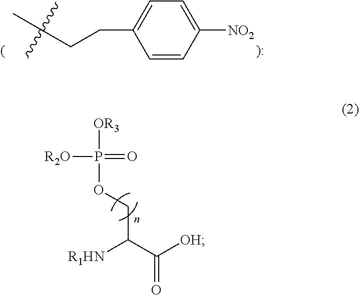
reducing compound 2 to produce compound 3: 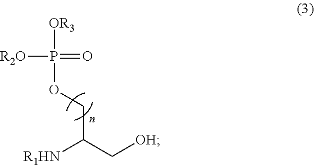
reacting compound 3 with Des s-Martin periodinane, followed by treating with NH₂CH₂C(O)OCH₃ and DCM, or reacting compound 3 with DMSO:TEA followed by treating with NH₂CH₂C(O)OCH₃, to produce compound 4: 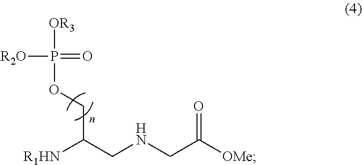
and
conjugating compound 4 with a nucleobase by reacting compound 4 with RCH₂C(O)OH, where R is a nucleobase in which α-amines of R are protected with an amine-protecting group to produce compound 5:

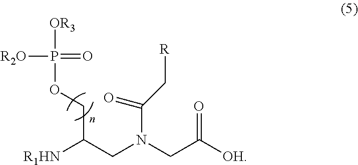

2. The method of claim 1, in which compound 1 is: 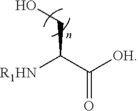
and compound 5 is:

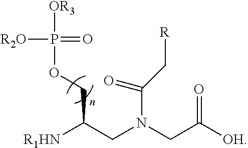

3. The method of claim 1, in which compound 1 is: 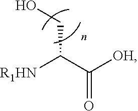
and compound 5 is:

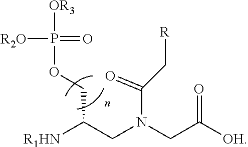

4. The method of claim 1, wherein n is 1.

5. The method of claim 1, wherein n is 2.

6. The method of claim 1, wherein R₁ Fmoc.

7. The method of claim 1, wherein R is adenine, thymine, guanine, cytosine, or uracil.

8. The method of claim 1, wherein R is a non-natural nucleobase.

9. (canceled)

10. A method of making a peptide nucleic acid monomer, comprising:
reducing compound 6: 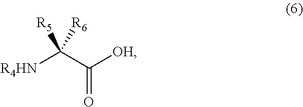
where R₄ is an amine-protecting group, and one of R₅ and R₆ is H, and the other of R₅ and R₆ is: 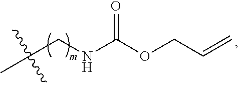
where m is 1, 2, 3, or 4,
to produce compound 7: 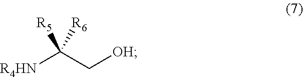
reacting compound 7 with Dess-Martin periodinane, followed by treating with NH₂CH₂C(O)OCH₃ (methyl glycinate), or reacting compound 7 with DMSO:TEA followed by treating with NH₂CH₂C(O)OCH₃, to produce compound 8: 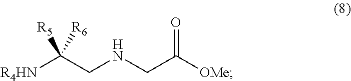
conjugating compound 8 with a nucleobase by reacting compound 8 with RCH₂C(O)OH, where R is a nucleobase in which α-amines of R are protected with an amine-protecting group to produce compound 9: 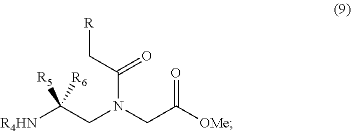
and
Removing the terminal methyl group of 9 to produce compound 10:

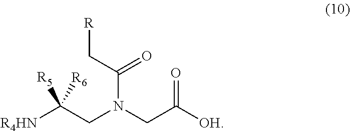

11. The method of claim 10, wherein R₅ or R₆ is H and the other of R₅ or R₆ is: 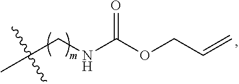
where m is 1, 2, 3, or 4.

12. (canceled)

13. The method of claim 10, wherein R₄ is Fmoc.

14. The method of claim 10, wherein m is 4.

15. (canceled)

16. The method of claim 10, wherein R is adenine, thymine, guanine, cytosine, uracil, or a non-natural nucleobase.

17. (canceled)

18. The method of claim 10, further comprising deprotecting the amine of: 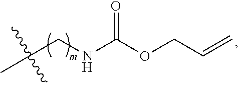
with Pd(PPh₃) or PhSiH₃, to produce compound 11: 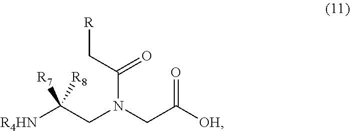
where one of R₇ and R₈ is H, and the other of R₇ and R₈ is 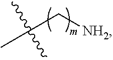
wherein m is 1, 2, 3, or 4.

19. A compound having the structure: 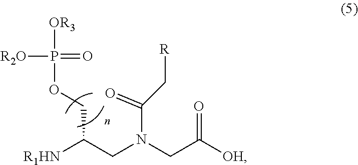
where R₂ and R₃ are, independently, H, benzyl, t-butyl, propionitrilyl, or 4-nitrophenylethylenyl, or a salt thereof.

20. The compound of claim 19, having the structure: 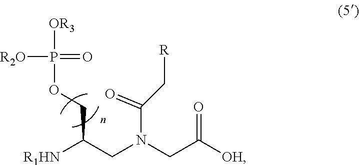
or a salt thereof.

21. The compound of claim 19, having the structure: 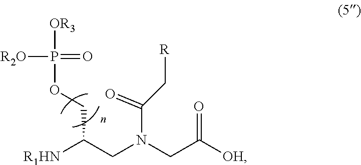
or a salt thereof.

22. (canceled)

23. (canceled)

24. A peptide nucleic acid comprising a residue of a monomer of the compound of claim 19.

25-45. (canceled)


SEQUENCE LISTING (2 sequences)

SEQ ID NO: 1 - RNA, 10 nt, Artificial Sequence
  Control RNA
  misc_feature (5)..(5): n is a, c, g, or u
ugaanuccau

SEQ ID NO: 2 - RNA, 10 nt, Artificial Sequence
  Control RNA
auggacuuca